(19) United States
(12) Patent Application Publication
(10) Pub. No.: US 2009/0188023 A1
(43) Pub. Date: Jul. 30, 2009

(54) Air Vent Eyeglasses Construction

(76) Inventor: Roger Wei Yi Hsu, Rancho Cucamonga, CA (US)

Correspondence Address: AGNEW INTERNATIONAL;PATENT & TRADEMARK LAW FIRM, 1300 BRISTOL STREET NORTH, SUITE 100, NEWPORT BEACH, CA, 92660, US

(21) Appl. No.: 12/258,367

(22) Filed: Oct. 24, 2008

Related U.S. Application Data
(60) Provisional application No. 60/982,198, filed on Oct. 24, 2007.

(51) Int. Cl. A61F 9/02

(52) U.S. Cl. 2/436; 2/440; 2/442; 2/445

(57) ABSTRACT

This invention discloses sports eyeglasses containing an inner removable sealing member, commonly referred to as eye cup, where the inner removable sealing member contains overlapping profile where the overlapping profile acts as attachment means to attach the inner removable sealing member to the main eyeglasses frame.

### CROSS REFERENCE TO RELATED APPLICATION

This application claims the benefit of priority under 35 U.S.C. 119(e) to the filing date of U.S. provisional patent application No. 60/982,198 entitled “Air Vent Sports Eyeglass Construction” which was filed Oct. 24, 2007, and is incorporated herein by reference.

### FIELD OF INVENTION

This present invention relates generally to eyewear. In particular, it relates to eyewear for use in sports and the like; and more particularly to eyewear which contains a removable sealing member.

### BACKGROUND OF THE INVENTION

Usually sports eyeglasses containing an inner removable sealing member where the inner removable sealing member is designed to be attached to the back of the main eyeglasses frame. In particular, the inner sealing member is designed to contain clips or the likes that attaches to the back of the main eyeglasses frame. More particularly, the clips of the inner sealing member are designed to be snapped into grooves formed in the back of the main eyeglasses frame.

Typically, where the side to which the inner removable sealing member attaches to the back side of the main eyeglasses frame (the side to which the lens is concaved) is considered as the front side of the inner removable sealing member, the back side of the inner removable sealing member is typically coupled with soft substances like foam or sponges so the inner removable sealing member when worn can wrap around the user's face and sufficiently seal against the skin of the face of the wearer from dusts and the likes.

In this typical construction, the combined the thickness of the side profile (not including the eyeglasses temple) of the entire eyeglasses construction (the frame, the inner sealing member and the foam or sponges) reaches 4 millimeter-12 millimeter, measuring from the center of the lens (as in the context of side profile) to the point of contact between the skin and the soft substances when the eyeglasses is worn.

There are several disadvantages to this typical construction. In particular, because the extended thickness of the side profile, the center of gravity of the entire eyeglasses (the main eyeglasses frame, the inner sealing member, and the sponges or foam) construction typically rests far from the eyes of the wearer when worn. Consequently, it causes the entire eyeglasses construction to shift forward when worn. This is undesirable because when the center of gravity rests far from the eyes of the wearer, it has a tendency to cause the eyeglasses to shift forward when worn and as a result the view to the side of the wearer is severely reduced. In addition, the eyeglasses is more likely to shift and eventually fallen off from the wearer when in use. Moreover, gaps are often created between the wearer's skin and the soft substances such as foam or sponge as the entire eyeglasses construction shifts forward during use, defeating the purpose of preventing dusts from coming into contact with the eyes of the wearer. Furthermore, with such extended distance, even blank lens can cause plus or minus degree.

### OBJECT OF THE INVENTION

It is an object of this invention to provide an eyeglasses construction wherein the eyeglasses containing removable sealing unit designed to fit in place between the eyeglasses and a wearer's face wherein the side profile of the eyeglasses construction is reduced by at least 1 mm to 14 mm as compare to the other eyeglasses construction in the market utilizing an overlapping profile design incorporated into the removable sealing unit wherein the overlapping profile attaches its extended profile to the eyeglasses.

It is an object of this invention to provide an eyeglasses construction wherein eyeglasses containing removable sealing unit designed to fit in place between the eyeglasses and a wearer's face wherein the center of gravity of the eyeglasses construction is close in proximity to the wearer's eyes wherein the eyeglasses is not likely to shift and fallen off from the wearer when in use.

### SUMMARY OF INVENTION

The present invention involves a sport eyeglasses frame where the inner sealing member is designed to contain an overlapping profile where the overlapping profile acts as attachment means such as clips, connectors, fasteners, and joinders to attach the inner removable sealing member to the main eyeglasses frame where the clips are designed to snap into the grooves formed on the front side of the main eyeglasses frame thus reducing the side profile (not including the temple of the eyeglasses) of the entire eye glass construction (the main eyeglasses frame, the inner sealing member, and the soft substances such as foam or sponge) by 1 millimeter to 5 millimeter.

In one embodiment the over lapping profile of the inner sealing member is acting as attachment means such as clips where grooves are formed on the front side of the eyeglasses frame over the bridge area of the main eyeglasses frame and the overlapping profile snaps into the grooves.

Another embodiment is for the overlapping profile of inner sealing member to act as holding means where grooves are formed on the outside of the temple of the eyeglasses frame and the overlapping profile acting as clips snap into the grooves formed on the outside of the temple of the eyeglasses frame. In this particular embodiment, the inside of the temple of the eyeglasses frame is the side to which that contacts the skin of the wearer and the outside of the temple of the eyeglasses frame is the opposite side to the inside of the temple of the eyeglasses frame.

In another embodiment the overlapping profile of the inner sealing member acts as attachment means over the main eyeglasses frame and the overlapping profile is secured to the main eyeglasses frame with screws or the likes.

In yet another embodiment the over lapping profile of the inner sealing member is acting as attachment means such as clips where grooved are formed on the front side of the main eyeglasses frame and over the top of lens of the main eyeglasses frame and the overlapping profile snaps into the grooves.

In yet another embodiment the overlapping profile of the inner sealing member is acting as attachment means such as clips where grooves are formed on the front side of the main eyeglasses frame and under the lens of the main eyeglasses frame and the overlapping profile snaps into the grooves.

In another embodiment the overlapping profile of the inner sealing member acts as attachment means and attach around the entire eyeglasses frame.

In yet another embodiment the inner sealing member is constructed in such way where the back side of the inner sealing member contains equally spaced risers where the attaching soft substances like foams or sponges are designed to contain equally spaced grooves to match the risers so that when the foam or sponge are attached to the back of the inner sealing member the riser acts as support and prevent the foam or sponge from collapsing after extended use.

In another embodiment the soft substance attached to the back of the inner sealing member is not air permeable.

In another embodiment the inner sealing member is made with durable plastic foam material made of ethylene vinyl acetate resin. Likewise, the inner sealing member can also be manufactured using materials such as plastic, rubber, or silicon.

In yet another embodiment, the inner sealing member also contains optical lens and the optical lens are removable.

In a preferred embodiment, the grooves formed on the outside of the eyeglasses frame for the overlapping profile of the inner sealing member to snap into additional function as air vents. In particular, the frame contains grooves formed on the outside of the main eyeglasses frame and around the lens where the grooves additional act as air vents, where air permeable material such as sponge, air permeable textile, and or wire mesh is placed inside the grooves, and where through holes are formed on the main eyeglasses frame inside the grooves, and where the inner sealing member also contains matching through holes when the inner sealing member is attached to the main eyeglasses frame. In this construction, air may pass from the outside of the eyeglasses through the air vents, the through holes, and the soft substances to the skin of the face. In this embodiment, the grooves are designed to be large enough where when overlapping profile snaps into the grooves there is sufficient room left in the grooves for the air to pass through.

In yet another embodiment, the through holes are placed around the inside edges of the corresponding lens opening of the inner sealing member. In this particular embodiment, the inner sealing member does not contain optical lens and only the main frame eyeglasses contains optical lens.

### DETAILED DESCRIPTION OF THE INVENTION

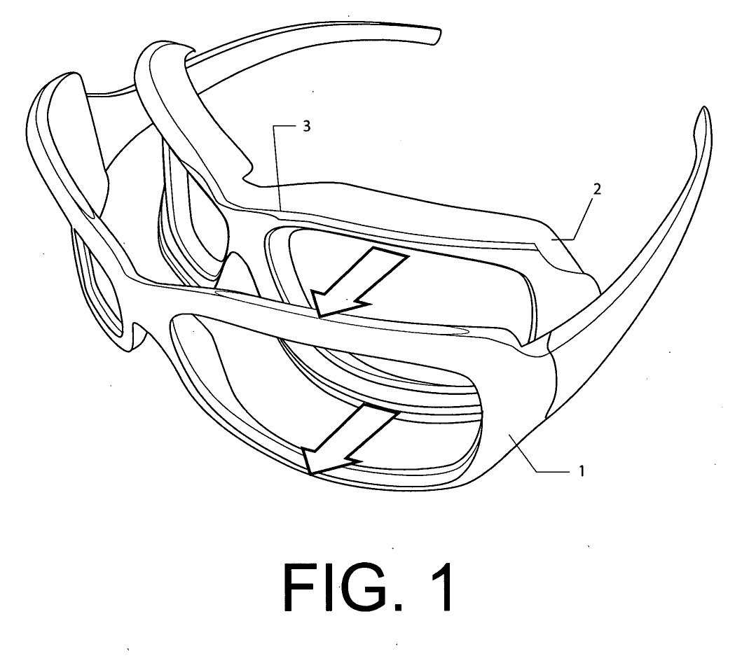

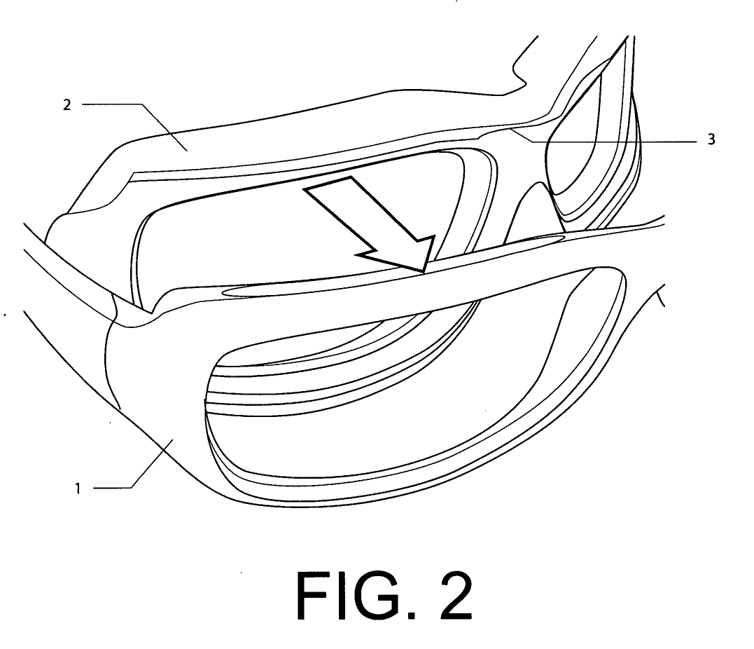

As shown in FIG. 1, eyeglasses of present invention is depicted. This includes a main eyeglasses frame 1, and an inner sealing member 2. FIG. 1 also depicts the inner sealing member 2 as containing an overlapping profile 3 on the top of the inner sealing member 1 where the overlapping profile is used as an attaching mean such as clips to the main frame 1. FIG. 2 is another depiction of the present invention where the inner sealing member 2 is shown to contain the overlapping profile 3 on the top of the inner sealing member 2 where the over lapping profile is used as an attaching mean to the main frame 1. In this particular embodiment, the overlapping profile 3 acts as a clip to fasten the inner sealing member 2 to the top of the main eyeglasses frame 1.

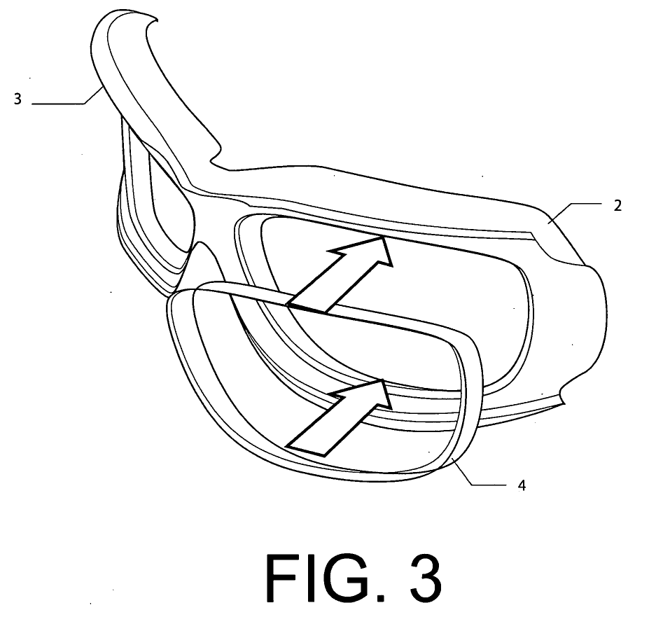

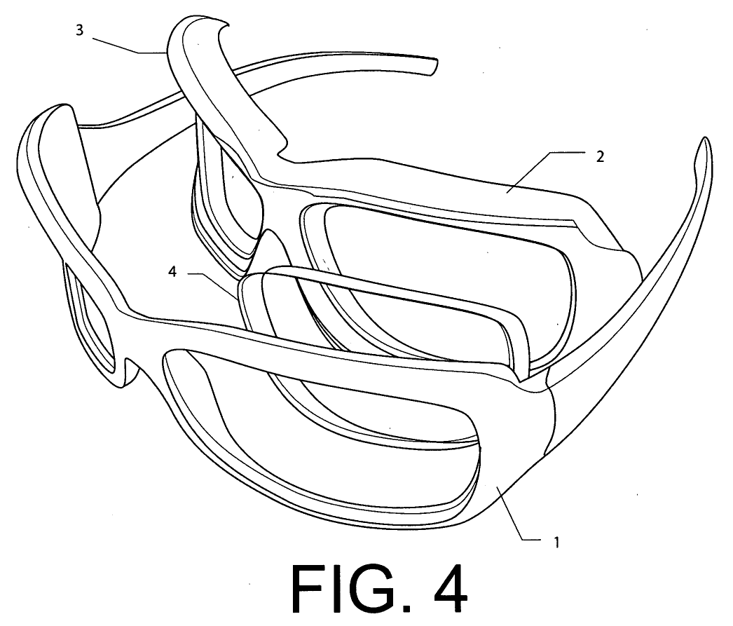

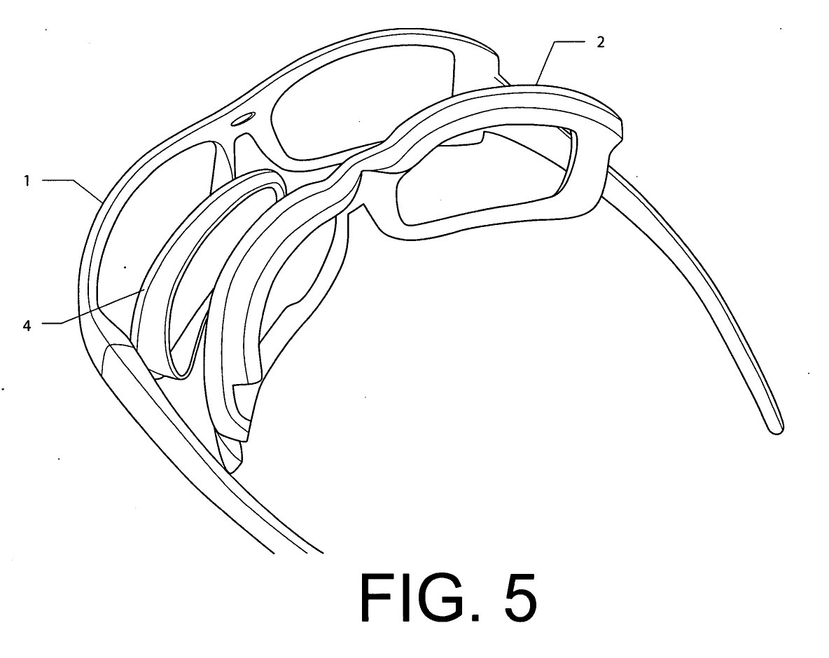

As shown in FIG. 5, the inner sealing member 2 contains optical lens 4 and the optical lens 4 are removable. Also shown in FIG. 3 is the overlapping profile 3 of the inner sealing member 2. As shown in FIG. 4, the main eyeglasses frame 1 is depicted along with the inner sealing member 2 and the overlapping profile 3 and the removable optical lens 4.

FIG. 5 is another depiction of FIG. 4 where the main eyeglasses frame 1 is depicted along with the removable optical lens 4 and the inner sealing member 2.

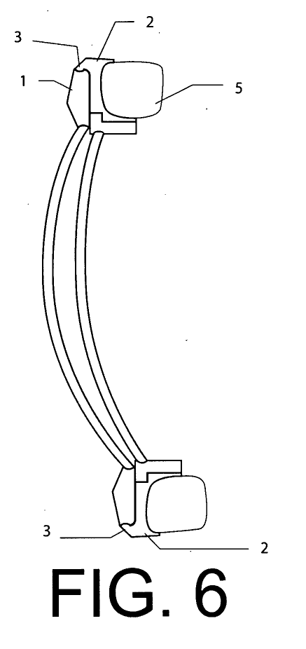

In FIG. 6, the dissected view of the side of the entire sports eyeglasses construction (less the temple) is depicted. In this depiction, the drawing discloses the main eyeglasses frame 1 being coupled with the inner sealing member 2 where the overlapping profile 3 of the inner sealing member 2 is used as clips to attach the inner sealing member 2 to both the top and the bottom of the main eyeglasses frame 1. Also depicted is the soft substance such as foam or sponge 5 used to attach to the back side of the inner sealing member.

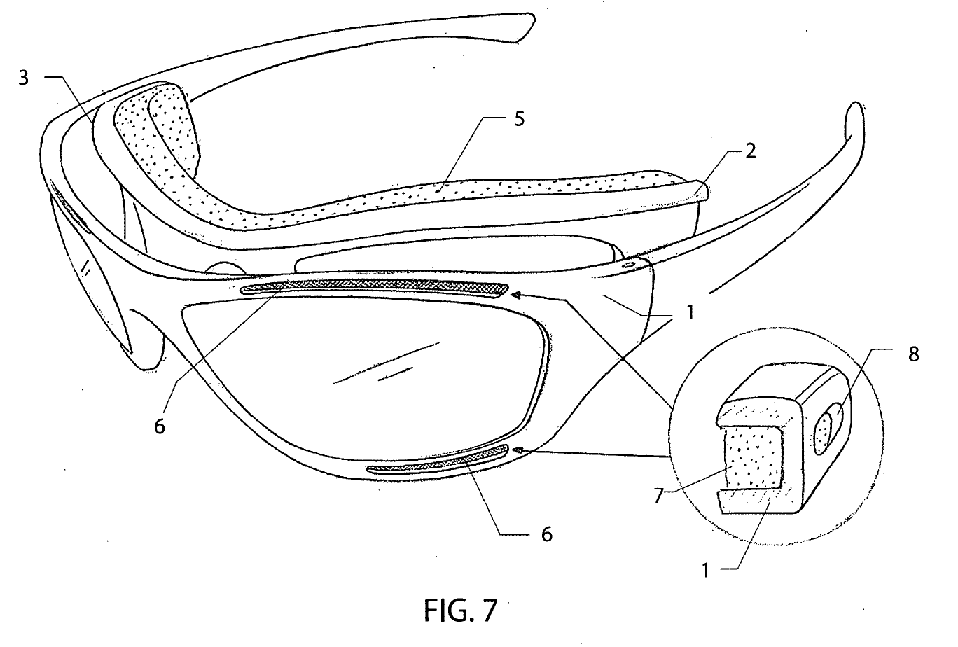

In FIG. 7, another preferred embodiment is disclosed. Here the main eyeglasses frame 1 is disclosed with air vents 6 on the top and bottom of the main eyeglasses frame 1 where an inner sealing member 2 is disclosed with its overlapping profile 3 and the soft substance such as foam or sponge 5 on the back of the inner sealing member. FIG. 7 further depicts air permeable material 7 such as wire mesh, sponge or foam being placed inside the air vent and through holes 8 on the main eyeglasses frame inside the air vents for air filtration.

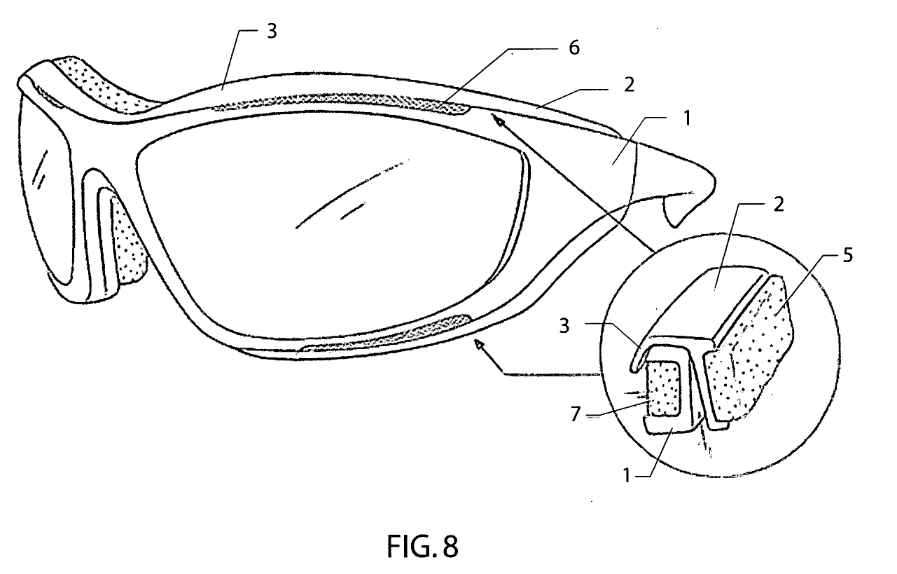

In FIG. 8, the main eyeglasses frame 1 is depicted to be attached with the inner sealing member 2 where the overlapping profile 3 is disclosed in detail as how it may be used as clips to attach to the main eyeglasses frame 1. Air vents 6 is also disclosed here in addition to the air permeable materials 7 placed inside the air vents 6. Further, FIG. 8 discloses foam or sponge material 5 as attached to the back of the inner sealing member.

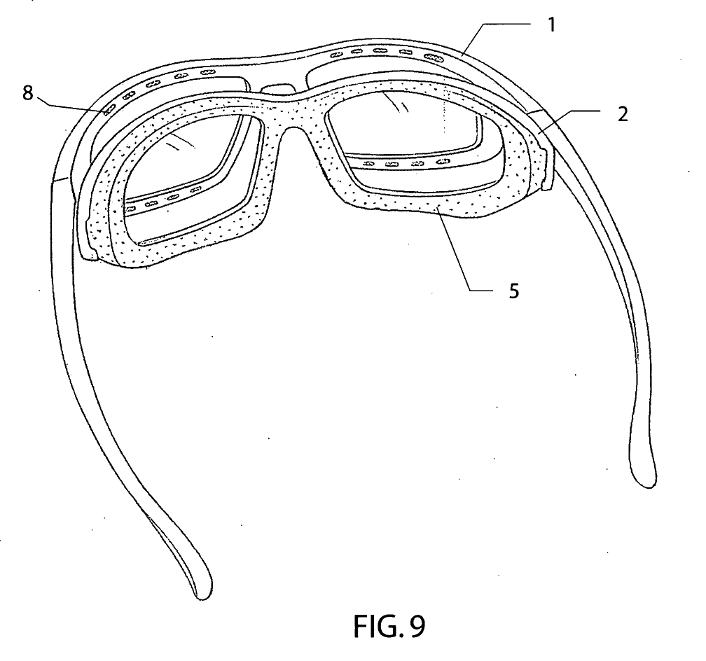

In FIG. 9, the through holes 8 as depicted in FIG. 7 are disclosed from another perspective. Here the through holes 8 are disclosed as they are placed on the main eyeglasses frame 1.

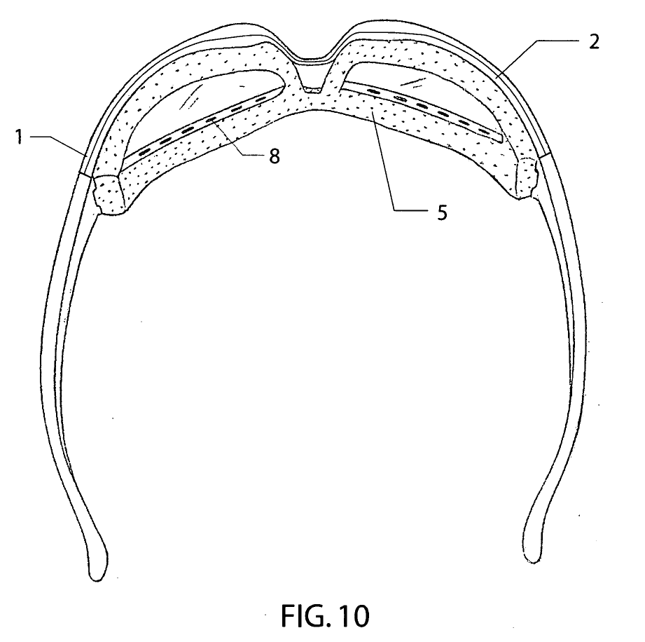

In FIG. 10, the through holes 8 are depicted as being placed around the inside edges of the corresponding lens opening of the inner sealing member 2.

I claim:

1. An eyeglasses frame, comprising a main eyeglasses frame having a left frame part and a right frame part, each of said frame parts comprising lens retaining portion, a bridge element; and an inner sealing member, where said inner sealing member comprises an overlapping profile, wherein said overlapping profile acts as an attachment device to attach said inner sealing member to the main eyeglasses frame wherein said inner sealing member is removable from said main eyeglasses frame.

2. The eyeglasses frame of method 1, wherein said overlapping profile attaches said inner sealing member to said main eyeglasses frame by clipping said overlapping profile onto one or more groves formed on said main eyeglasses frame.

3. The eyeglasses frame of method 2 wherein said grooves are formed on the front side of said main eyeglasses frame.

4. The eyeglasses frame of method 2 wherein said grooves are formed on a front side of said main eyeglasses frame.

5. The eyeglasses frame of method 2 wherein said grooves are formed on a front side over a bridge area of said main eyeglasses frame.
The eyeglasses frame of method 2 wherein said grooves are formed on the outside of the temple of said main eyeglasses frame.

6. The eyeglasses frame of method 4 wherein said grooves are further formed on the top of said right frame part and said left frame part of said main eyeglasses frame.

7. The eyeglasses frame of method 6 wherein said grooves are further formed on the bottom of said right frame part and said left frame part of said main eyeglasses frame.

8. The eyeglasses frame of method 1, wherein said overlapping profile attaches said inner sealing member to said main eyeglasses frame by clipping said overlapping profile onto the entire edges of said main eyeglasses frame.
The eyeglasses frame of method 1, wherein said inner sealing member is constructed wherein its back side contains equally spaced risers wherein an attached soft substances like foams comprises equally spaced grooves to match said equally spaced risers wherein when said are attached to the back of said inner sealing member said risers act as support and prevent said foam from collapsing after an extended use.

9. The eyeglasses frame of method 1, wherein said overlapping profile attaches said inner sealing member to said main eyeglasses frame by clipping said overlapping profile onto the entire edges of said main eyeglasses frame.
The eyeglasses frame of method 1, wherein said inner sealing member's back side contains equally spaced risers wherein an attached soft substances like foam comprises equally spaced grooves to match said equally spaced risers wherein when said soft substances like foam is attached to said inner sealing member's back side said risers act as structure support for said foam and prevent said foam from collapsing after an extended use.

10. The eyeglasses frame of method 9, wherein said foam is not air permeable.

11. The eyeglasses frame of method 1 wherein said inner sealing member is made with durable plastic foam material of ethylene vinyl acetate resin.

12. The eyeglasses frame of method 1 wherein said inner sealing member is made with durable material selected from the group consisting of plastic, rubber, and silicon.

13. The eyeglasses frame of method 1 wherein said inner sealing member contains optical lens.

14. The eyeglasses frame of method 13 wherein said optical lens are removable.

15. The eyeglasses frame of method 2, wherein said grooves act as air vents wherein said grooves contain air permeable material wherein through holes are formed inside said grooves wherein said air vents facilitate air passage through said through holes.

16. The eyeglasses frame of method 1, wherein said overlapping profile attaches said inner sealing member to said main eyeglasses frame wherein said overlapping profile is secured to said main eyeglasses frame by attachment device.

17. The eyeglasses frame of method 16, wherein said attachment device is selected from a group consisting of screws, nails, and fasteners.
